(19) United States
(12) Patent Application Publication
(10) Pub. No.: US 2023/0279510 A1
(43) Pub. Date: Sep. 7, 2023

(54) QUANTIFICATION OF CORONAVIRUS RNAAEMIA

(71) Applicants: Universite Paris Cite, Paris (FR); Assistance Publique - Hopitaux de Paris, Paris (FR); Centre National de la Recherche Scientifique, Paris (FR); Inserm (Instilut National de la Sante et de la Recherche Medicale), Paris (FR); Sorbonne Universite, Paris (FR)

(72) Inventors: Helene Pere, Paris (FR); David Veyer, Paris (FR); Valerie Taly, Bourg-la-Reine (FR); Pierre Laurent-Puig, Meudon (FR)

(21) Appl. No.: 18/007,667

(22) PCT Filed: May 31, 2021
(86) PCT No.: PCT/EP2021/064575

§ 371 Date: Dec. 1, 2022

(30) Foreign Application Priority Data
Jun. 1, 2020 (EP) 20305571.0

(51) Int. Cl. C12Q 1/70

(52) U.S. Cl. CPC C12Q 1/701; C12Q 2600/118; C12Q 2600/106; C12Q 2600/158

(57) ABSTRACT

The invention relates to methods for quantifying a SARS coronavirus in a blood sample of a subject, and for determining a risk for the subject to develop a severe form of the disease or for the disease to worsen.

The present invention relates to methods for quantifying a SARS coronavirus in a blood sample of a subject, and for determining a risk for the subject to develop a severe form of the disease or for the disease to worsen.

### TECHNICAL BACKGROUND

Three coronaviruses have crossed the species barrier to cause deadly pneumonia in humans since the beginning of the 21st century: severe acute respiratory syndrome coronavirus (SARS-CoV), Middle-East respiratory syndrome coronavirus (MERS-CoV), and SARS-CoV-2. SARS-CoV-2, was discovered in December 2019 in Wuhan, Hubei province of China. On Jan. 30, 2020, the World Health Organization declared the SARS-CoV-2 epidemic a public health emergency of international concern.

SARS-CoV-2, responsible for the development of COVID-19, was first isolated and sequenced in early January 2020 (Chen Y 2020; Chen L 2020). Although most patients present mild-to-moderate disease, 5 to 10% progress to severe or critical disease (Huang C 2020), including pneumonia and acute respiratory failure. Based on data from patients with laboratory-confirmed COVID-19 from mainland China, admission to intensive care unit (ICU), invasive mechanical ventilation or death occurred in 5.0%, 2.3% and 1.4% of cases, respectively (Guan W-J 2020). In severe cases, clinical observations typically describe a two-step disease progression, starting with a mild-to-moderate presentation followed by a secondary respiratory worsening 9 to 12 days after onset of first symptoms (Grasselli G 2020; Huang C 2020; Li Q 2020). At the time of pulmonary deterioration, although the clinical manifestations of SARS-CoV-2 are dominated by respiratory symptoms, patients may suffer severe systemic damage such as cardiovascular, renal injury and liver injury and/or multiple organ failure (Huang C 2020; Yang F 2020). In addition, coagulopathy has been reported in severe COVID-19 cases (Han 2020; Tang 2020). These systemic clinical features strongly suggest the spread of SARS-CoV-2 within extra-pulmonary sites via the blood flow.

The semi-quantitative detection of circulating SARS-CoV-2 RNA as assessed by conventional real-time reverse transcription-polymerase chain reaction assay (RT-PCR) used for routine SARS-CoV-2 molecular diagnosis from respiratory tract samples was previously reported in COVID-19 patients in few studies (Chen X 2020; Chen W 2020; Huang C 2020). However determinations by real-time RT-PCR are indirect and provide a relative quantification of viral load. The precision of such assays is also limited by the nature of cycle threshold (Ct) determination with resulting values having a relative standard deviation that can exceed 50%. The same RT-PCR technology was used to detect RNA serum SARS-CoV-2 RNA in a retrospective limited series of 6 COVID-19 patients (Chen W 2020).

### SUMMARY OF THE INVENTION

The inventors now provide quantitative detection of circulating SARS RNA (RNAaemia) in blood samples.

The inventors have more particularly shown that the level of circulating SARS-CoV-2 viral load increased with the severity of COVID-19 and the proportion of detectable SARS-CoV-2 RNAaemia significantly correlated with disease severity.

On that basis, the inventors provide a method for determining a risk for a subject to develop or show an aggravation of, a SARS coronavirus-induced acute pulmonary failure and/or systemic damage, which method comprises measuring the quantity of circulating coronavirus RNA (RNAaemia) in a sample of the subject, wherein the sample is a blood, plasma or serum sample of the subject.

It is also provided a method for classifying a subject infected with SARS coronavirus, which method comprises measuring the quantity of circulating coronavirus RNA in a sample of the subject, wherein the sample is a blood, plasma or serum sample of the subject.

It is further provided a method for monitoring efficacy of a therapeutic treatment against a SARS coronavirus infection or SARS coronavirus-induced acute pulmonary failure and/or systemic damage in a subject, which method comprises measuring the quantity of circulating coronavirus RNA (RNAaemia) in a sample of the subject, wherein the sample is a blood, plasma or serum sample of the subject collected at different points of time before, and during and/or after the subject has been subjected to the therapeutic treatment.

### LEGENDS TO THE FIGURES

FIGS. 1A and 1B show plasmatic SARS-CoV-2 RNAaemia (cp/mL) measured by droplet-based digital PCR according to clinical classes.

FIG. 2: Kaplan-Meier curves showing the prognostic value of circulating SARS-CoV-2 RNAaemia as assessed by frequency of degradation events at time of plasma viral detection by droplet-based digital PCR. The grey and hatched black lines represent patients without and with detectable SARS-CoV-2 RNAaemia, respectively. The hatched line at 0.5 represents the median survival.

### DETAILED DESCRIPTION OF THE INVENTION

### Definitions

The “subject” or “patient” to be treated may be any mammal, preferably a human being. The human subject may be a child, an adult or an elder. The subject has been infected with a SARS coronavirus. In a preferred embodiment, the subject has been diagnosed with COVID-19 and/or have been tested positive with a SARS-CoV-2 RT-PCR testing on a respiratory sample (e.g. a nasopharyngeal swab or an invasive respiratory sample).

The term “infection by a SARS coronavirus” includes any stage of infection. In particular embodiments, the coronavirus may be SARS-CoV, and SARS-CoV-2, preferably SARS-CoV-2, responsible for the COVID-19 pandemic.

As used herein, the term “treatment” or “therapy” includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. In the context of the present invention, a curative treatment more particularly refers to reducing the risk of worsening of the disease, especially COVID-19. Particularly, the treatment may aim at preventing a mild or moderate stage ARDS from developing to a more severe stage (in reference to the Berlin definition, defined below), and ultimately at preventing death of the patient. Prophylactic treatment refers to any of: halting the onset, reducing the risk of development of disease, reducing the incidence, delaying the onset, reducing the development as well as increasing the time to onset of symptoms of a particular disorder. In the context of the present invention, the prophylactic treatment more particularly refers to preventing or minoring symptoms of the infection by the coronavirus, in particular preventing the disease, especially COVID-19, to develop and to trigger an ARDS.

A patient is diagnosed with a hypoxemia typically when his/her PaO₂/FiO₂ ratio is at most 300 mmHg for a positive tele-expiratory pressure set at minus 5cmH₂O (PaO2 represents the arterial partial pressure of dioxygen and FiO2 is the inspired fraction of dioxygen in the gas inhaled by the patient).

An acute respiratory distress syndrome (ARDS) is a condition that results from an attack on the alveolo-capillary membrane leading to so-called “lesional” pulmonary edema. The 2012 Berlin conference (Ranieri et al. 2012,) defined ARDS by 4 criteria: 1) an onset of acute respiratory symptoms within 7 days of an alveolar attack, 2) hypoxemia which results in a PaO₂/FiO₂ ratio≤300 mmHg for a positive tele-expiratory pressure set at minus 5cmH₂O, 3) the presence of bilateral pulmonary opacities in chest imaging, 4) pulmonary edema which is not explained by the preferential increase in hydrostatic pressure. The ARDS case fatality rate is inversely associated with the value of the PaO₂/FiO₂ ratio.

TABLE 1
Berlin Definition of ARDS. Ranieri et al, 2012

| | |
|---|---|
| Timing | Within 1 week of a known clinical insult or new or |
| | worsening respiratory symptoms |
| Chest imaging^{a} | Bilateral opacities- not fully explained by effusions, |
| | lobar/lung collapse, or nodules |
| Origin of edema | Respiratory failure not fully explained by cardiac |
| | failure or fluid overload |
| | Need objective assessment (e.g. echocardiography) to |
| | exclude hydrostatic edema if no risk factor present |
| Oxygenation^{b} | |
| MILD | 200 mm Hg < PaO₂/FiO₂ ≤ 300 mm Hg with PEEP or |
| | CPAP ≥ 5 cm H₂O^{c} |
| MODERATE | 100 mm Hg < PaO₂/FiO₂ ≤ 200 mm Hg with PEEP or |
| | CPAP ≥ 5 cm H₂O |
| SEVERE | PaO₂/FiO₂ ≤ 100 mm Hg with PEEP or CPAP ≥ 5 |
| | cm H₂O |

Abbreviations CPAP, continuous positive airway pressure; FiO₂, fraction of inspired oxygen; PaO₂, partial pression of arterial oxygen; PEEP, positive end-expiratory pressure.
^{a}chest radiograph or computed tomography scan.
^{b}if altitude is higher than 1000 m, the correction factor should be calculated as follows [PaO₂/FiO₂ × (barometric pressure/760)].
^{c}this may be delivered noninvasively in the mild acute respiratory distress syndrome group.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR). A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification. A typical amplification reaction is carried out by contacting a forward and reverse primer (a primer pair) to the sample DNA together with any additional amplification reaction reagents under conditions which allow amplification of the target sequence.

RNA Extraction and Amplification

A sample of blood is obtained from a subject according to methods well known in the art. In the method of the invention, the RNA may be measured from a whole blood sample. In another preferred embodiment, the method makes use of plasma or serum samples.

Plasma or serum may be isolated according to methods known in the art.

As an example, RNA may be extracted from the blood, plasma or the serum immediately or within 1 hour, 2 hours, 3 hours, 4 hours, 5 hours or 6 hours. Optionally the plasma or serum can be stored after blood centrifugation at −20° C. or −80° c. prior to isolation of the RNA.

Methods of RNA extraction are well known in the art.

In a particular embodiment, the RNA from the sample may be fractionated prior to performing an amplification reaction. In a preferred embodiment, the amplification reaction is a digital droplet PCR reaction (ddPCR).

It is thus herein described a method for quantifying circulating coronavirus RNA (RNAaemia) in a sample of the subject, wherein the sample is a blood, plasma or serum sample of the subject, which method comprises extracting the RNA from the sample and amplifying the RNA by a reverse transcriptase digital droplet PCR.

Droplet-based digital PCR is based on the realization of thousands to millions of single-molecule PCRs in parallel in independent compartments and the resulting amplification products reflects more closely the original composition of nucleic acid mixtures than conventional PCR while in parallel being more tolerant to presence of inhibitors compared to bulk-based systems (Pekin D 2011; Perkins G 2017; Taly V 2012; Yu F 2020).

Digital PCR samples are partitioned into thousands of independent endpoint PCR reactions prior to amplification, and a reaction well is scored as either positive or negative for amplification of the viral sequence of interest. The positive wells are counted and converted to a concentration of target in the original sample. This binary assignment of each reaction greatly minimizes the measurement's dependency on parameters such as assay efficiency and instrument calibration. As a result, this enables different laboratories to compare viral load measurement results in a standardized manner without interference from external factors.

A description of this technology is provided e.g. in international patent application WO2015/013681, incorporated herein by reference.

To fractionate the RNA sample/specimen, emulsification techniques can be used so as to create large numbers of aqueous droplets that function as independent reaction chambers for the PCR reactions. For example, an aqueous specimen (e.g., 20 microliters) can be partitioned into droplets (e.g., 20,000 droplets of one nanoliter each) to allow an individual test for the target to be performed within each of the droplets.

Aqueous droplets can be suspended in oil to create a water-in-oil emulsion (W/O). The emulsion can be stabilized with a surfactant to reduce coalescence of droplets during heating, cooling, and transport, thereby enabling thermal cycling to be performed.

In an exemplary droplet-based digital assay, a specimen is partitioned into a set of droplets at a dilution that ensures that more than 40 percent, preferably more than 50, 60, 70, 80, or 90 percent of the droplets contain no more than one RNA molecule per specimen fraction.

Once fractionation has taken place, the RNA may then optionally be amplified.

The primers and probes used for amplification need not reflect the exact sequence of the target nucleic acid sequence (i.e. need not be fully complementary), but must be sufficiently complementary so as to hybridize to the target site under the particular experimental conditions. Accordingly, the sequence of the oligonucleotide typically has at least 70 percent homology, preferably at least 80 percent, 90 percent, 95 percent, 97 percent, 99 percent or 100 percent homology, for example over a region of at least 13 or more contiguous nucleotides with the target sequence. The conditions are selected such that hybridization of the oligonucleotide to the target site is favored and hybridization to the non-target site is minimized.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

Target Genes

The viral load, reflected by the RNAaemia, may be measured by quantifying any RNA that is specific to the coronavirus to test.

The genome of coronaviruses, ranging from 26 to 32 kilobases in length, includes a variable number of open reading frames (ORFs). The SARS-CoV-2 genome was reported to possess 14 ORFs encoding 27 proteins.

Among the major structural proteins, which include the spike surface glycoprotein (S), small envelope protein (E), matrix protein (M), and nucleocapsid protein (N), a preferred target gene for quantifying RNAaemia is the gene that encodes the N protein. In a particular embodiment, the methods involve measuring the absolute quantity of at least the RNA of the N gene, however any other RNA specific to the virus may be further quantified.

Correlation with Clinical Severity and Prediction of Aggravation

The method described herein allows to classify a subject infected with SARS coronavirus, according to the severity of the infection.

The method described herein further allows to determine the risk for a subject to develop, or show an aggravation of, a SARS coronavirus-induced acute pulmonary failure and/or systemic damage.

Such system damage includes e.g. cardiovascular injury, renal injury, liver injury and/or multiple organ failure.

All can lead to the death of the subject.

The higher the RNAaemia, the higher the severity of the condition and the risk for the subject. According to the invention, the quantification of circulating RNA is thus a marker of severity, as well as a marker for predicting the clinical development and outcome, or a marker for determining the risk for the subject to develop symptoms, or to show an aggravation of SARS coronavirus-induced acute pulmonary failure and/or systemic damage, and risk of death.

In a preferred embodiment, especially useful when the SARS coronavirus is SARS-CoV-2, the sample is collected between 5 to 15 days, preferably between 8 to 12 days, still preferably about 10 days, after first symptoms of the SARS-induced disease (respectively COVID-19), including e.g. fever and/or dry cough.

In another preferred embodiment, also especially useful when the SARS coronavirus is SARS-CoV-2, the sample is collected at DO and/or at different points of time between DO and D10, preferably at D0, D3, D6, D9, wherein
D0 (day 0) is the first day of symptoms, including e.g. fever and/or dry cough, and D10 is the tenth day after at least a first symptom has occurred, or
D0 (day 0) is the day wherein the subject has been diagnosed with a SARS virus infection, preferably wherein the subject has been tested positive with a SARS virus (e.g. SARS-CoV-2) RT-PCR testing on a respiratory sample (e.g. a nasopharyngeal swab or an invasive respiratory sample).

According to the method of the invention, an RNAamia that is negative at D0 and remains negative is indicative of a subject that has no or a low risk of aggravation. Conversely, an RNAamia that increases starting from D0 is indicative of a subject that is at risk of aggravation.

The method more particularly allows to determine whether patients with mild or moderate symptoms, including e.g. dyspnea, shortness of breath and respiratory distress, including any or several symptoms such as polypnea, cyanosis, grunting, nose flaring, sweating, wheezing, and/or chest retractions, are likely to develop aggravated symptoms, which would require an emergency care to avoid or delay developing into a severe or critical case. The method further allows to determine whether patients with severe symptoms are likely to worsen.

Patients can be typically classified as follows (based on an adaptation of the Sixth Revised Trial Version of the Novel Coronavirus Pneumonia Diagnosis and Treatment Guidance)

Mild cases: The clinical symptoms (e.g. fever, myalgia, fatigue, and/or diarrhea) are mild, and there is no sign of pneumonia on imaging, e.g. on thoracic computed tomography (CT) scan.

Moderate cases: Showing fever and respiratory symptoms (such as dyspnea) with radiological findings of pneumonia, e.g. on thoracic CT scan. Such patients generally require a maximum of 3 L/min of oxygen.

Severe cases: Cases meeting any of the following criteria: i) Respiratory distress (30 breaths/min); ii) Oxygen saturation≤93% at rest; iii) Arterial partial pressure of oxygen (PaO2)/fraction of inspired oxygen (FiO2)≤300 mmHg (1 mmHg=0.133 kPa). PaO2/FiO2 in high-altitude areas (at an altitude of over 1,000 meters above the sea level) shall be corrected by the following formula: PaO2/FiO2 x[Atmospheric pressure (mmHg)/760] Cases with chest imaging that showed obvious lesion progression within 24-48 hours>50% are managed as severe cases. Such patients generally require at least 3 L/min of oxygen with no other organ failure.

Critical cases: Cases meeting any of the following criteria: i) Respiratory failure and requiring mechanical ventilation; ii) Shock; and/or iii) With other organ failure that requires intensive care.

An RNAaemia below to 2 log₁₀ copies (cp)/ml (namely above about 100 copies/ml), preferably between 0 and 80 cp/ml, or 20 to 100 cp/ml, is typically indicative of a mild to moderate case.

An RNAaemia that is equal or superior to 2 log₁₀ copies (cp)/ml (namely above about 100 copies/ml), preferably more than about 150 cp/ml, is typically indicative of a severe case.

Furthermore an RNAaemia equal or superior to 250 cp/ml, preferably equal or superior to 300 cp/ml, e.g. equal or superior to 2.5 log₁₀ cp/mL is indicative of a subject at risk of aggravation or a critical case.

In a preferred embodiment, the subject shows hypoxemia. In a particular aspect the subject shows hypoxemia and the method is for use in assessing the risk of aggravation to a more severe stage of pulmonary failure and/or systemic damage, or death.

In still a particular embodiment, the subject has an ARDS, preferably a mild or moderate ARDS, and the method allows to determine the risk for the subject to show a severe ARDS.

In a particular aspect, an RNAaemia of 250 copies/mL or more is indicative of a subject likely develop a deterioration during his follow-up as acute pulmonary failure event, such as an acute respiratory distress syndrome (ARDS), and/or systemic damage, e.g. cardiovascular injury, renal injury, liver injury and/or multiple organ failure or death.

Monitoring of Treatment

The method described herein further allows monitoring efficacy of a therapeutic treatment against a SARS coronavirus infection or SARS coronavirus-induced acute pulmonary failure and/or systemic damage in a subject.

For that purpose, samples are collected in a patient who undergoes a candidate therapeutic treatment, at different points of time, preferably at least one time before initiation of the therapeutic treatment, and at least another time during the course of the treatment, or after the treatment. Assessing the sensitivity of a subject with respect to a candidate treatment makes it possible to adjust the dosage or regimen of the treatment or to change treatment.

In a preferred embodiment, the subject is at an early stage of the disease, e.g. they show early symptoms such as fever and/or dry cough.

The method more particularly allows to determine whether a patient with mild or moderate symptoms, including dyspnea, shortness of breath and respiratory distress, including any or several symptoms such as polypnea, cyanosis, grunting, nose flaring, sweating, wheezing, chest retractions, benefits from the candidate treatment.

Examples of candidate therapeutic treatments include antimicrobial agents such as antifungals or antibiotics, antiviral agents and/or immunomodulators.

Anti-microbial therapeutic agents of interest encompass, without being limited to, remdesevir, lopinavir, ritonavir, favipiravir, camostat mesylate, azithromycin, chloroquine, hydroxy-chloroquine.

Immunomodulators encompass, without being limited to, immunosuppressants such as cyclosporine, mycophenolate, anti-IL6, anti-ILL interferons, and/or biotherapies.

A stagnation or a decrease of the RNAaemia, preferably by at least 10%, 20%, or 30%, upon treatment indicates that the therapeutic treatment is effective in the subject. The stronger decrease, the better the prognosis for the subject.

An increase of the RNAaemia is indicative that the treatment is ineffective and of a bad prognosis. Such cases compel a change of treatment and an intensive care of the subject.

In another aspect it is herein described a method for treating a subject infected with a SARS coronavirus, especially for treating COVID-19, which method comprises quantifying the coronavirus RNAaemia, so as to assess the status of the subject and the risk of aggravation, and administering a therapeutic treatment to the subject, to fight against development and/or worsening of acute pulmonary failure, such as an acute respiratory distress syndrome (ARDS), and/or systemic damage, e.g. cardiovascular injury, renal injury, liver injury and/or multiple organ failure and/or death.

The following examples illustrate the invention without limiting its scope.

### Example 1: Quantification of Circulating SARS-CoV-2 Viral Load (RNAaemia) by Droplet-Based Digital PCR

Methods

Study Design and Patients

Patients admitted to Hôpital Cochin, Paris, France, at the time of respiratory deterioration and between days 8 and 12, were included between Mar. 19, 2020 and Apr. 3, 2020, in the setting of the local RADIPEM biological samples collection derived from samples collected in routine care. Biological collection and informed consent were approved by the Direction de la Recherche Clinique et Innovation (DRCI) and the French Ministry of Research (N°2019-3677). Inclusion criteria for COVID-19 inpatients were: age between 18 and 80 years old, diagnosis of COVID-19 according to WHO interim guidance, and positive SARS-CoV-2 RT-PCR testing on a respiratory sample (nasopharyngeal swab or invasive respiratory sample). In patients with pre-existing unstable chronic disorders (such as uncontrolled diabetes mellitus, severe obesity defined as body mass index greater than 30, unstable chronic respiratory disease or chronic heart disease) were excluded. Since median duration from onset of symptoms to respiratory failure was shown to be 9.5 (interquartile range, 7.0-12.5) days in our cohort, we analyzed SARS-CoV-2 RNAaemia on samples collected between 8 to 12 days after first symptoms.

Healthy controls were RT-PCR SARS-CoV-2 negative asymptomatic adults, matched with cases on age and sex.

The clinical severity of COVID-19 described according to the adaptation of the Sixth Revised Trial Version of the Novel Coronavirus Pneumonia Diagnosis and Treatment Guidance published on Feb. 19, 2020. Mild cases were defined as patients with mild clinical symptoms (fever, myalgia, fatigue, and diarrhea) and no sign of pneumonia on thoracic computed tomography (CT) scan. Moderate cases were defined as patients with clinical symptoms associated with dyspnea and radiological findings of pneumonia on thoracic CT scan, and requiring a maximum of 3 L/min of oxygen. Severe cases were defined as respiratory distress patients requiring aver 3 L/min of oxygen with no other organ failure. Critical cases were defined as patients requiring mechanical ventilation, into shock and/or with other organ failures that required intensive care unit (ICU) cares. The study conforms to the principles outlined in the Declaration of Helsinki, and received approval by the appropriate Institutional Review Board (Cochin-Port Royal Hospital, Paris, France; number AAA-2020-08018).

Quantification of SARS-CoV-2 RNAaemia

Plasma SARS-CoV-2 RNA (140 μL) was extracted using QIAamp® Viral RNA Mini Kit (QIAGEN®, Hilden, Germany), according to manufacturer's instructions. SARS-CoV-2 RNAaemia was quantified at each time point by droplet-based Crystal Digital PCR™ (Stilla Technologies, Villejuif, France) on the Naica™ System (Stilla Technologies, Villejuif, France), which includes primers and FAM- and HEX-labeled probes specific to two distinct regions [ORF1ab and Nucleocapside (N) genes] of the SARS-CoV-2 positive strand RNA genome. The 3^{rd} channel of the Naica™ system was used as an endogenous PCR control detecting a human housekeeping gene with a Cy5-labeled probe. This single assay design permits the simultaneous detection of two independent SARS-CoV-2 sequences reported as conserved, while concurrently monitoring PCR effectiveness using the third channel of detection. Plasma samples with one of the two ORF1 or N genes or both genes detected were considered as positive samples and results were automatically analyzed using “Crystal reader” (Stilla) and “Crystal Miner” software (Stilla Technologies) based on the most amplified gene positive droplets. SARS-CoV-2 RNA concentrations (cp/mL) were finally calculated considering the extracted volume of plasma.

Statistical Analysis

Descriptive statistics were computed for the population at baseline. Quantitative variables were described as mean±standard deviation (SD) if normally distributed, or median and inter-quartile range (IQR) otherwise. Categorical variables were described as group sizes and percentages.

Bivariate comparisons between clinical classes were computed using the Fisher test for categorical variables and the Kruskal-Wallis one-way analysis of variance for continuous variables. When comparing control and COVID-19 patients, the Mann-Whitney rank-sum test was used for quantitative variables.

An ordinal regression multivariate model was used to evaluate the association between the clinical class and circulating viral DNA quantification (log values), adjusted for clinical predictors. We simply described the patients' clinical outcome using Kaplan-Meier curves and we did not perform statistical survival analysis, as this study was not designed for survival analysis, the patients belonging to different clinical classes at baseline, and the outcome being a composite criterion of mechanical ventilation requirement or death.

Computations were performed using the R software, and the ordinal package for the ordinal regression model. P values<0.05 were considered significant.

Results

Patients Characteristics

Fifty-eight COVID-19 patients and 12 healthy controls were analyzed. Demographic and clinical characteristics of the patients are shown in Table 2 below. Median age of the patients was 55.1 years (interquartile range, 15) and 81% were male.

On admission, the degree of severity of COVID-19 was categorized as mild-to-moderate in 17 patients (median oxygen requirement 2 L/min), severe in 16 patients (median oxygen requirement 5 L/min) and critical in 26 patients. All of 55 CT scan available at time of admission were abnormal, showing ground-glass opacities (100%) with bilateral patchy distribution (95%). No patients with mild-to-moderate disease required admission to ICU or use of mechanical ventilation, while 5 out of 15 patients with severe disease were admitted to ICU. No healthy controls and 55% of COVID-19 patients did not present any comorbidity. In the remaining COVID-19 patients (45%), the controlled comorbidities described were non-insulin diabetes mellitus (n=7), solid cancers (n=6), hematological cancer (n=3), asthma (n=3), autoimmune diseases (n=3) and chronic obstructive pulmonary disease (n=1). Fifteen COVID-19 patients received an antiviral treatment at time of their hospitalization for disease progression.

TABLE 2
Clinical characteristics of hospitalized COVID-19 patients

| | N | % | Med | IQR |
|---|---|---|---|---|
| Sexe | 58 | | | |
| Female | 11 | 19 | | |
| Male | 47 | 81 | | |
| Age | 58 | | 55.14 | 15 |
| Clinical classes | 58 | | | |
| Mild/Moderate | 17 | 29 | | |
| Severe | 15 | 26 | | |
| Critical | 26 | 45 | | |
| Clinical Degradation | 58 | | | |
| Death | 3 | 5.2 | | |
| Mechanical Ventilation | 3 | 5.2 | | |
| Optiflow | 3 | 5.2 | | |
| Absence | 49 | 84 | | |
| Monitoring Delay | 58 | | 7 | 11 |
| Pulmonary radiography showing ground-glass | 55 | | | |
| opacities | | | | |
| No | 0 | 0 | | |
| Yes | 55 | 100 | | |
| Pulmonary radiography showing bilateral | 55 | | | |
| patchy distribution | | | | |
| No | 3 | 5.5 | | |
| Yes | 52 | 95 | | |
| Controlled comorbidities | 58 | | | |
| No | 32 | 55 | | |
| Yes | 26 | 45 | | |
| Oxygeno-requerance | 58 | | | |
| No | 3 | 5 | | |
| Yes | 29 | 50 | | |
| Mechanical Ventilation | 26 | 45 | | |
| Mechanical Ventilation | 26 | 45 | | |
| Antiviral treatment | 58 | | | |
| No | 43 | 74 | | |
| Yes | 15 | 26 | | |

IQR: Interquartile range

SARS-CoV-2 RNAaemia Results by ddPCR

At time of clinical deterioration, 43 (74.1%) of COVID-19 patients were positive for SARS-CoV-2 RNA in plasma, with at least the N gene detected. 28% were positive for ORF1 gene, then systematically associated with N gene positivity.

Proportion of positive SARS-CoV-2 RNAaemia was significantly different between patients from different clinical classes, from 53% in mild-to-moderate patients to 88% in critically ill patients (p=0.036) (Table 2).

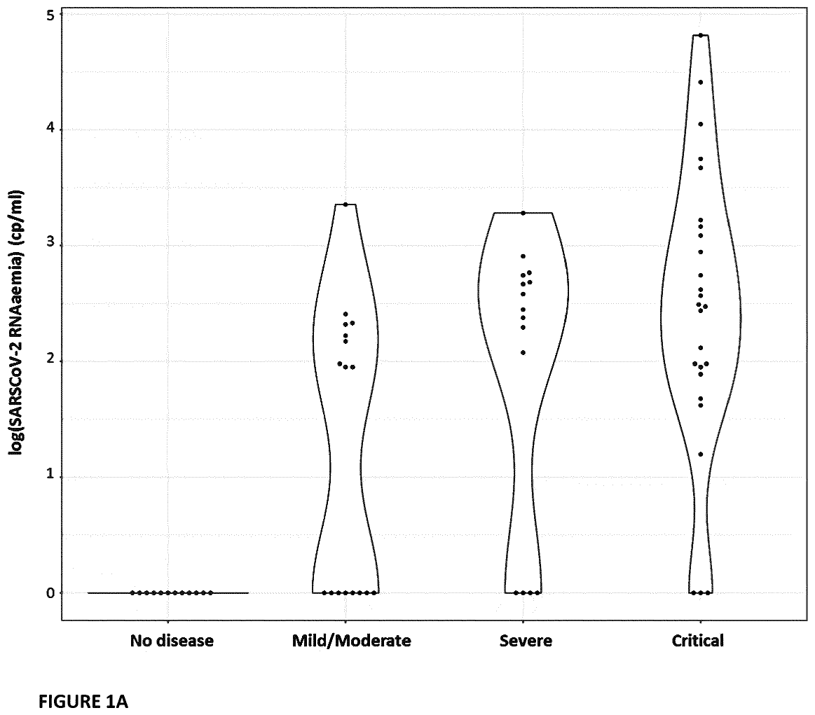

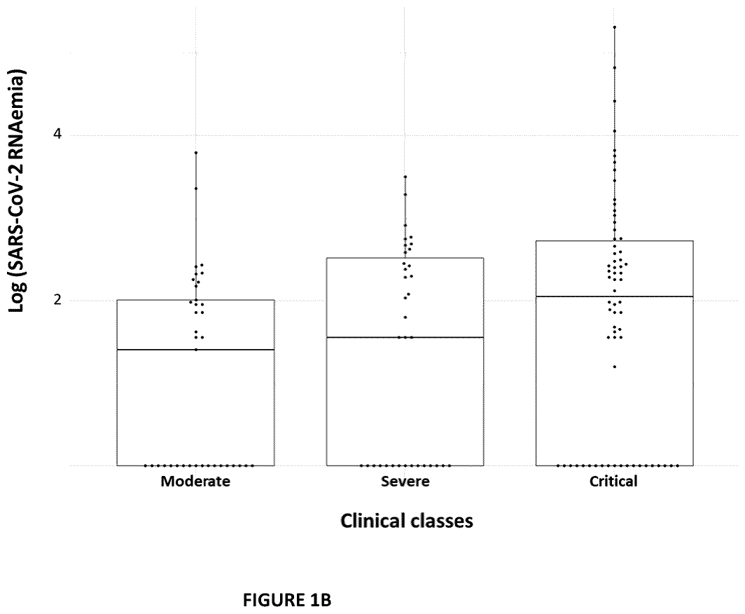

SARS-CoV-2 RNAaemia also correlated with COVID-19 severity (p=0.035), as depicted in FIG. 1A, with median SARS-CoV-2 RNAaemia of 89, 279 and 301 cp/mL, in mild-to-moderate, severe and critically ill patient groups, respectively.

In multivariate analysis adjusted on age, sex and comorbidities, SARS-CoV-2 RNAaemia was strongly associated with the clinical class (adjusted hazard ratio 2.2, 95% CI 1.39-3.9; p=0.0018) (Table 3 below).

All healthy controls showed negative SARS-CoV-2 RNAaemia with no detection of both specific SARS-CoV-2 N and ORF-1 genes.

TABLE 3
Univariate and multivariate analysis of demographic, clinical and virological data

| | Clinical classes | | | | | | | | | | | | Univariate | Multivariate | |
|---|---|---|---|---|---|---|---|---|---|---|---|---|---|---|---|
| | Mild/moderate | | | | Severe | | | | Critical | | | | analyses | analyses | |
| | N | % | Median | IQR | N | % | Median | IQR | N | % | Median | IQR | P | OR | P |
| Sex | 17 | | | | 15 | | | | 26 | | | | 0.52 | 2.6[0.63-11] | 0.19 |
| Female | 5 | 29 | | | 2 | 13 | | | 4 | 15 | | | | | |
| Male | 12 | 71 | | | 13 | 87 | | | 22 | 85 | | | | | |
| Age | 17 | | 51 | 24 | 15 | | 57.54 | 12 | 26 | | 55.16 | 12 | 0.24 | 1[0.98-1.1] | 0.23 |
| Clinical Degradation | 17 | | | | 15 | | | | 26 | | | | 0.00016 | | |
| Death | 0 | 0 | | | 0 | 0 | | | 3 | 12 | | | | | |
| Mechanical Ventilation | 0 | 0 | | | 3 | 20 | | | 0 | 0 | | | | | |
| Optiflow | 0 | 0 | | | 3 | 20 | | | 0 | 0 | | | | | |
| Absence | 17 | 100 | | | 9 | 60 | | | 23 | 88 | | | | | |
| Controlled comorbidities | 17 | | | | 15 | | | | 26 | | | | 0.0021 | 7.3[2.2-29] | 0.0023 |
| No | 15 | 88 | | | 8 | 53 | | | 9 | 35 | | | | | |
| Yes | 2 | 12 | | | 7 | 47 | | | 17 | 65 | | | | | |
| Positive SARSCoV-2 | 17 | | | | 15 | | | | 26 | | | | 0.036 | | |
| RNAaemia | | | | | | | | | | | | | | | |
| No | 8 | 47 | | | 4 | 27 | | | 3 | 12 | | | | | |
| Yes | 9 | 53 | | | 11 | 73 | | | 23 | 88 | | | | | |
| SARSCOV-2 RNAaemia | 17 | | 89 | 167 | 15 | | 279.76 | 458 | 26 | | 303.57 | 1318 | 0.015 | 2.2[1.4-3.9] | 0.0018 |
| (cp/mL) | | | | | | | | | | | | | | | |
| Log(SARSCoV-2 | 17 | | 2 | 2.2 | 15 | | 2.45 | 1.7 | 26 | | 2.48 | 1.2 | | | |
| RNAaemia) (cp/mL) | | | | | | | | | | | | | | | |

OR: Odd ratio;
IQR: Interquartile range

Clinical Monitoring and Correlation with Baseline SARS-CoV-2 RNAaemia

A total of nine events of clinical deterioration was observed during the follow-up of the COVID-19 patients: respiratory deterioration from respiratory distress requiring oxygen using optiflow (n=3) and mechanical ventilation (n=3) in severe patients and death for critical patients (n=3).

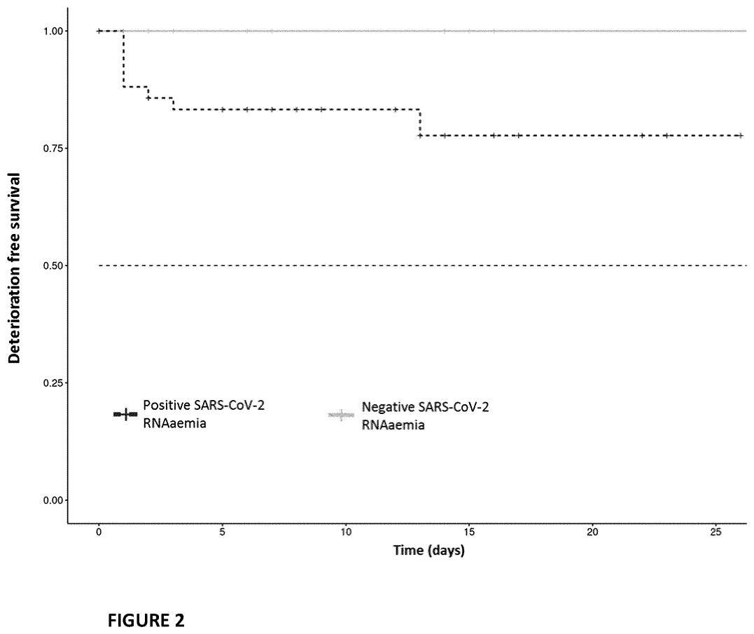

Eight of the nine COVID-19 patients with clinical deterioration belonged to patients with positive SARS-CoV-2 RNAaemia, while only one critical patient with undetectable SARS-CoV-2 RNAaemia at the time of analysis died at day 27 (FIG. 2). Of note, the patient with the highest SARS-CoV-2 RNAaemia (65,476 cp/ml) died from COVID-19 one day after plasma sampling.

In the present study, SARS-CoV-2 RNAaemia was measured by ddPCR in a large cohort of hospitalized COVID-19 patients at time of disease worsening. SARS-CoV-2 RNAaemia was detectable in the majority of patients, confirming that the SARS-CoV-2 may invade the systemic compartment beyond the lungs.

Circulating SARS-CoV-2 viral load showed a large amplitude, spanning almost 4 login, with a significant association between viral load levels and clinical deterioration. The proportion of patients with detectable SARS-CoV-2 RNAaemia was also strongly correlated with disease severity. Furthermore, viraemic COVID-19 patients showed a tendency to have a higher risk of poor outcome, unlike non-viraemic patients.

### Example 2: Quantification of Circulating SARS-CoV-2 Viral Load (RNAaemia) by Droplet-Based Digital PCR in Merged Cohorts

To complete and extend the observations of SARS-CoV-2 plasmatic RNAaemia quantified by ddPCR as biomarker of COVID-19 clinical severity and outcome, another series of 79 patients hospitalized for COVID-19 was retrospectively included during the first wave of the epidemic in France between March and July 2020. In this global study bringing together two cohorts of comparable COVID-19, the absolute quantification of SARS-CoV-2 RNAaemia was reported in the plasma of 139 COVID-19 hospitalized patients. The interest of this marker was evaluated in specifying the degree of clinical severity of COVID-19, and correlated with the clinical outcome by assessing the degree of systemic viral invasion in COVID-19 patients at hospital admission.

Study design and patients. Two cohorts of respectively 60 COVID-19 patients admitted to the Cochin Hospital, Paris, France, and 79 to the European George Pompidou Hospital (HEGP), Paris, France, were included between Mar. 19, 2020 and Jun. 26, 2020. Inclusion criteria for COVID-19 inpatients were: age between 18 and 80 years, diagnosis of COVID-19 according to World Health Organization (WHO) interim guidance, and positive SARS-CoV-2 RT-PCR testing on a respiratory sample (nasopharyngeal swab or invasive respiratory sample). The clinical severity of COVID-19 described according to the adaptation of the Sixth Revised Trial Version of the Novel Coronavirus Pneumonia Diagnosis and Treatment Guidance published on Feb. 19, 2020.

Mild cases, moderate cases, severe cases and critical cases were as defined in Example 1.

Quantification of Plasmatic RNAaemia

The levels of SARS-CoV-2 RNAaemia were quantified as described in Example 1.

Statistical Analysis

Descriptive statistics were computed for the population at baseline. Quantitative variables were described as mean±standard deviation (SD) if normally distributed, or median and inter-quartile range (IQR) otherwise. Categorical variables were described as group sizes and percentages.

Bivariate comparisons between clinical classes were computed using the Fisher test for categorical variables and the one-way analysis of variance for continuous variables when all groups were normally distributed and the Kruskal-Wallis one-way analysis of variance otherwise. When comparing control and COVID-19 patients, the Student t test was used for quantitative variables when both groups were normally distributed, and the Mann-Whitney rank-sum test otherwise.

The Cox proportional hazards model was used to evaluate the risk of death at inclusion between patients with low and high plasmatic RNAse P concentration.

Patients' clinical outcomes are presented using Kaplan-Meier curves. We did not perform a formal statistical survival analysis, since the study was not primarily designed for survival analysis and the patients belonging to different clinical classes at baseline.

Computations were performed using the R software, and the survival package for the Cox proportional hazards model. P values<0.05 were considered significant.

Results

Patient characteristics. 139 hospitalized COVID-19 patients were included. Patients were 59 years old on average (SD=14) and 78% were male.

Patients in both cohorts were globally comparable, although the Cochin Hospital cohort patients were slightly younger (mean age 55 vs 62) and presented less comorbidities (30% vs 53% hypertension, 3% vs 15% chronic renal failure) at inclusion than patients in the HEGP cohort.

At inclusion, the degree of severity of COVID-19 was categorized as mild-to-moderate in 37 (27%) patients, severe in 35 (25%) patients and critical in 67 (48%) patients. The median time from symptom onset to plasma sample was of 11 days in the global merged cohort (respectively 10 days and 13 days for Cochin Hospital and HEGP cohort). Seventy-six (54%) of the COVID-19 patients do not present any comorbidities. In the remaining COVID-19 patients (63, 46%), comorbidities were: diabetes (n=29; 25/29 are isolated non-insulin diabetes mellitus), cardiovascular history (n=21), hypertension (n=60), cancer history (n=13) and chronic renal failure (n=14).

SARS-CoV-2 RNAaemia Results by ddPCR

Correlation between SARS-CoV-2 RNAaemia determined by ddPCR and clinical classes. These results confirm that ddPCR quantification of SARS-CoV-2 RNAaemia in COVID-19 patients at hospital admission is significantly correlated with clinical severity (Table 4; Kruskal-Wallis test P=0.021; FIG. 1B).

TABLE 4
SARS-COV-2 RNAaemia concentrations in 139 patients suffering
from COVID-19 according to clinical severity

| | Moderate | | | Severe | | | Critical | | | |
|---|---|---|---|---|---|---|---|---|---|---|
| | N | Med | IQR | N | Med | IQR | N | Med | IQR | p |
| SARS-COV-2 | 37 | 25 | 101 | 35 | 36 | 330 | 66 | 113 | 528 | 0.021 |
| RNAaemia | | | | | | | | | | |
| (cp/mLplasma) | | | | | | | | | | |
| log(SARS-COV-2 | 37 | 1.4 | 2 | 35 | 1.6 | 2.5 | 66 | 2 | 2.7 | 0.021 |
| RNAaemia) | | | | | | | | | | |

### REFERENCES

Chen L et al, Early Transmission Dynamics in Wuhan, China, of Novel Coronavirus-Infected Pneumonia. N. Engl. J. Med. 2020, 382, 1199-1207
Chen X, et al. Detectable serum SARS-CoV-2 viral load (RNAaemia) is closely correlated with drastically elevated interleukin 6 (IL-6) level in critically ill COVID-19 patients. Clin Infect Dis 2020; published online April 17. DOI:10.1093/cid/ciaa449.
Chen Y et al,. Emerging coronaviruses: Genome structure, replication, and pathogenesis. J. Med. Virol. 2020; 92: 418-23.
Chen W et al, Emerging Microbes & Infections, 2020, VOL. 9, 469-473
Grasselli G et al, COVID-19 Lombardy ICU Network, Baseline Characteristics and Outcomes of 1591 Patients Infected With SARS-CoV-2 Admitted to ICUs of the Lombardy Region, Italy. JAMA, 2020, 323(16):1574-1581
Guan et al, China Medical Treatment Expert Group for Covid-19, Clinical Characteristics of Coronavirus Disease 2019 in China. N. Engl. J. Med, 2020; 382:1708-1720
Han et al. Prominent changes in blood coagulation of patients with SARS-CoV-2 infection. Clin Chem Lab Med 2020; published online March 16. DOI:10.1515/cclm-2020-0188.
Huang C et al. Clinical features of patients infected with 2019 novel coronavirus in Wuhan, China. Lancet 2020; 395: 497-506.
Li Q et al. Early transmission dynamics in Wuhan, China, of novel coronavirus-infected pneumonia. N Engl J Med 2020; 382:1199-207.
Pekin, et al, Quantitative and sensitive detection of rare mutations using droplet-based microfluidics. Lab on a chip (2011), 11(13): 2156-66.
Perkins, et al, Droplet-based digital PCR: application in cancer research. Advanced in clinical Chemistry (2017), 79: 43-91.
Ranieri et al, Acute respiratory distress syndrome: the Berlin Definition. JAMA. 2012; 307(23):2526-33
Taly, et al, Detecting Biomarkers with microdroplet technology. Trends in Molecular Medicine. 2012 July; 18(7):405-16.
Tang et al. Abnormal coagulation parameters are associated with poor prognosis in patients with novel coronavirus pneumonia. J Thromb Haemost 2020; 18: 844-7.
Yang F et al. Analysis of 92 deceased patients with COVID-19. J Med Virol 2020; published online April 15. DOI:10.1002/jmv.25891.
Yu et al. Quantitative Detection and Viral Load Analysis of SARS-CoV-2 in Infected Patients. Clin Infect Dis 2020; published online March 28. DOI:10.1093/cid/ciaa345.

1. A method for determining a risk for a subject to develop or show an aggravation of, a SARS coronavirus-induced acute pulmonary failure and/or systemic damage, which method comprises measuring the quantity of circulating coronavirus RNA (RNAaemia) in a sample of the subject, wherein the sample is a blood, plasma or serum sample of the subject.

2. The method of claim 1, wherein the SARS coronavirus is SARS-CoV-2.

3. The method of claim 1, wherein the sample has been collected between 8 to 12 days after first symptoms of a SARS coronavirus infection.

4. The method of claim 1, wherein the subject shows hypoxemia.

5. The method of claim 1, wherein the sample has been collected at D0 and/or at different points of time between D0 and D10, preferably at D0, D3, D6, D9, wherein
D0 (day 0) is the first day of symptoms of a SARS-induced disease or is the day wherein the subject has been diagnosed with a SARS coronavirus infection.

6. The method of claim 1, wherein an RNAaemia of 2,5 log₁₀ copies/ml or more is indicative of a subject likely develop an acute pulmonary failure, such as an acute respiratory distress syndrome (ARDS), and/or systemic damage, e.g. cardiovascular injury, renal injury, liver injury and/or multiple organ failure, or to die.

7. The method of claim 1, wherein the subject shows hypoxemia and the method is for use in assessing the risk of aggravation to a more severe stage of pulmonary failure and/or systemic damage.

8. A method for classifying a subject infected with SARS coronavirus, which method comprises measuring the quantity of circulating coronavirus RNA (RNAaemia) in a sample of the subject, wherein the sample is a blood, plasma or serum sample of the subject.

9. The method of claim 7, wherein the SARS coronavirus is SARS-CoV-2.

10. The method of claim 8, wherein an RNAaemia of 2,5 log₁₀ copies/ml or more is indicative of a severe case.

11. A method for monitoring efficacy of a therapeutic treatment against a SARS coronavirus infection or SARS coronavirus-induced acute pulmonary failure and/or systemic damage in a subject, which method comprises measuring the quantity of circulating coronavirus RNA (RNAaemia) in a sample of the subject, wherein the sample is a blood, plasma or serum sample of the subject collected at different points of time before, and during and/or after the subject has been subjected to the therapeutic treatment.

12. The method of claim 11, wherein the SARS coronavirus is SARS-CoV-2.

13. The method of claim 11, wherein a decrease of the RNAaemia by at least 10% upon treatment indicates that the therapeutic treatment is effective in the subject.

14. The method of claim 1, wherein the RNA is N gene RNA.

15. The method of claim 1, wherein the RNAaemia is measured by a digital probe-based RT-PCR, preferably a digital droplet RT-PCR.

16. The method of claim 8, wherein the RNA is N gene RNA.

17. The method of claim 8, wherein the RNAaemia is measured by a digital probe-based RT-PCR, preferably a digital droplet RT-PCR.

18. The method of claim 11, wherein the RNA is N gene RNA.

19. The method of claim 11, wherein the RNAaemia is measured by a digital probe-based RT-PCR, preferably a digital droplet RT-PCR.
